(19) United States
(12) Patent Application Publication
(10) Pub. No.: US 2007/0244315 A1
(43) Pub. Date: Oct. 18, 2007

(54) Process for the preparation of cefdinir

(76) Inventors: Vinod Kumar Kansal, Faridabad (IN); Dhirenkumar N. Mistry, Gujarat (IN); Saurabh Pandey, Uttar Pradesh (IN); Rakesh Patel, Gujarat (IN); Shlomit Wizel, Petah Tiqva (IL)

Correspondence Address: KENYON & KENYON LLP, ONE BROADWAY, NEW YORK, NY, 10004, US

(21) Appl. No.: 11/591,246

(22) Filed: Oct. 31, 2006

Related U.S. Application Data
(60) Provisional application No. 60/732,097, filed on Oct. 31, 2005.

(51) Int. Cl. C07D 501/02

(52) U.S. Cl. 540/219

(57) ABSTRACT

The invention relates to processes for preparing cefdinir via its potassium and cesium salts.

### RELATED APPLICATIONS

This application claims the benefit of priority to U.S. Provisional Application No. 60/732,097, filed Oct. 31, 2005, the contents of which are herein incorporated by reference.

### FIELD OF THE INVENTION

The invention relates a process for preparing cefdinir via its potassium and cesium salts.

### BACKGROUND OF THE INVENTION

Cefdinir is a third generation cephalosporin antibiotic for oral administration and has a broader antibacterial spectrum over general gram positive and gram negative bacteria than other antibiotics for oral administration. Cefdinir, currently marketed as OMNICEF®, is an antibiotic prescribed in a 300 mg oral capsule or a suspension of 125 mg/5 mL. OMNICEF® is prescribed for respiratory and ear infections. Cefdinir is otherwise known as 7-(Z)[2-(2-aminothiazol-4-yl)-2-hydroxyiminoacetimido]-3-vinyl-3-cephem-4-carboxylic acid and has the following structure:

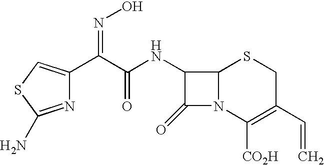

Examples 14 and 16 of U.S. Pat. No. 4,559,334 disclose the synthesis of cefdinir. In example 14, cefdinir is obtained by reacting benzhydryl 7-(4-bromoacetoacetamido)-3-vinyl-3-cephem-4-carboxylate in dichloromethane and acetic acid with isoamyl nitrite at −3° C. to −5° C., followed by addition of acetylacetone. Thiourea was added and the benzyhydryl group was cleaved with trifluoroacetic acid. After work up, the organic layer was acidified and cooled at 0° C. to obtain the crystalline cefdinir. Compound 9 of example 2 discloses the sodium salt of cefdinir.

U.S. Pat. No. 4,935,507 discloses two methods of obtaining crystalline cefdinir. Crystalline cefdinir may be crystallized from methanol to obtain crystalline cefdinir Form A. Alternatively the crystalline form is stepwise purified. In the stepwise process, the amorphous form was dissolved in water, washed with saturated sodium bicarbonate, acidified, passed by column chromatography, and treated with activated charcoal. The pH of the resultant solution was adjusted to 1.8 at 35° C. and the resultant crystalline cefdinir was collected. The cefdinir obtained in the '507 patent is cefdinir Form A. The '507 patent shares one common inventor with the '334 patent and the same assignee. The '507 patent characterizes the product of examples 14 and 16 of the '334 patent as a crystalline like amorphous product, not a crystalline product. The '507 patent further states “the amorphous product has disadvantages that it is bulky, not so pure, unstable and insufficient in filtration rate, therefore it is not suitable for a pharmaceutical product or is not easy to handle in pharmaceutical preparations, in producing it in a large scale or in storage.”

US Publication No. 2003/0204082 describes a process for preparing crystalline cefdinir at a temperature between 0° C. and +6° C. from a dilute aqueous solution of cefdinir in the presence of at least one organic solvent, in a total percentage (volume (v) to volume (v) on the aqueous solution) not exceeding 10% and at a pH between 1.5 and 3. The cefdinir obtained in the '082 application is said to be cefdinir Form B.

U.S. Pat. No. 6,093,814 describes processes for preparing cefdinir that entail the use of an intermediate having a trityl protecting group and one molecule of p-tolunenesulfonic acid and two molecules of N,N-dimethylacetamide attached to the main structure of the intermediate. Neither cefdinir potassium nor cefdinir cesium is described in the '814 patent.

PCT publication WO 98/45299 discloses a cefdinir dicyclohexylamine salt and mentions that cefdinir may be purified via the dicyclohexylamine salt.

PCT Publication WO 02/098884 describes preparing cefdinir by “treating a cefdinir intermediate with a formic acid-sulfuric acid mixture or a formic acid-methanesulfonic acid mixture to obtain a crystalline salt of cefdinir and reacting the crystalline salt with a base in a solvent.”

PCT publication WO 03/050124 describes a novel crystalline cefdinir potassium dihydrate, a process for its preparation and its use for the preparation of cefdinir.

US publication No. 2004/0242556 discloses a crystalline form of cefdinir, a process to prepare it and its use in pharmaceutical compositions.

The synthesis of a complex organic molecule such as cefdinir is challenging in that many steps are involved, with each step affecting the quantity and quality of the final product obtained. Although methods of synthesizing cefdinir and salts thereof are known, there is a continuing need for economically advantageous processes that yield cefdinir in high purity and/or with fewer steps.

### SUMMARY OF THE INVENTION

In one embodiment, the invention encompasses a process for preparing cefdinir comprising: reacting a protected thioester of Formula I: 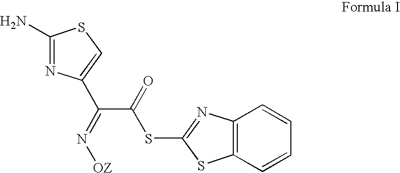
, 7-amino-3-vinyl-3-cephem-4-carboxylic acid and at least one organic base in the presence of water and a water-miscible organic solvent to form protected cefdinir; converting the protected cefdinir to a cefdinir salt selected from the group consisting of cefdinir potassium and cefdinir cesium; and converting the cefdinir salt to cefdinir; wherein Z represents an oxime protecting group.

Preferably, the process comprises: reacting a thioester of Formula I 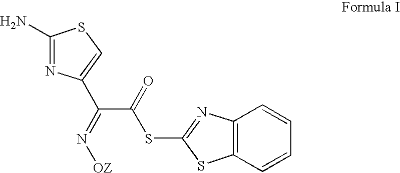
with 7-amino-3-vinyl-3-cephem-4-carboxylic acid and at least one organic base in the presence of water and a water-miscible organic solvent to obtain a reaction mixture comprising protected cefdinir; treating the reaction mixture with a base in the presence of a buffer and a source of potassium or cesium ions to obtain cefdinir potassium salt or cesium salt to form a mixture; recovering the salt from the mixture; dissolving the salt in water or a mixture of water and a water miscible organic solvent to form a solution; and adding an acid to the solution to obtain cefdinir, wherein Z represents an oxime protecting group.

### DETAILED DESCRIPTION OF THE INVENTION

In one embodiment, the invention relates to a process for preparing cefdinir comprising:
(a) reacting a protected thioester of Formula I: 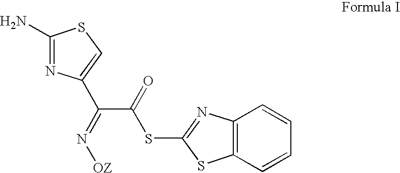
, 7-amino-3-vinyl-3-cephem-4-carboxylic acid and a organic base in the presence of water and a water-miscible organic solvent to form protected cefdinir;
(b) converting the protected cefdinir to a cefdinir salt selected from the group consisting of cefdinir potassium and cefdinir cesium; and
(c) converting the cefdinir salt to cefdinir;

wherein Z represents an oxime protecting group.

Preferably, the cefdinir salt obtained in step b) is recovered prior to step c).

Preferably, the process includes: reacting the thioester of Formula I with the 7-amino-3-vinyl-3-cephem-4-carboxylic acid and at least one organic base in the presence of water and a water-miscible organic solvent to obtain a reaction mixture comprising protected cefdinir, treating the reaction mixture with a base in the presence of a buffer and a source of potassium or cesium to obtain cefdinir potassium salt or cesium salt respectively, recovering the salt, dissolving the salt in water or a mixture of water and a water missible organic solvent to form a solution, and adding an acid to the solution to obtain cefdinir, wherein Z represents an oxime protecting group.

Preferably, the oxime protecting group in the compound of formula I and the protected cefdinir is selected from the group consisting of: acetyl, 2-amino thiazole and tetrahydropyranyl.

Preferably, the water-miscible organic solvent is selected from the group consisting of: tetrahydrofuran, ethanol, methanol, propanol, isopropanol, N, N dimethyl formamide, dimethyl acetamide, acetonitrile and mixtures thereof. More preferably, the water-miscible organic solvent is tetrahydrofuran. Preferably, the ratio of the water-miscible solvent to water is about 1:1 to about 10:1 (v:v), more preferably, about 2.5:1 (v:v).

Preferably, the organic base is an amine base. Preferably, the amine base is a C₃ to C₁₂ amine, more preferably, the amine base is a C₃-C₉ amine. Preferably, the C₃-C₁₂ amine is selected from the group consisting of: diethylamine, triethylamine, diisopropylamine, tri-n-butylamine, triethylenediamine, and pyridine. More preferably, the C₃-C₉ amine is triethylamine.

The reaction mixture comprising the protected cefdinir may be stirred. Preferably, the reaction mixture is stirred for about 2 to about 8 hours, more preferably, for about 4 to about 6 hours. A suitable temperature for the reaction is about 0° C. to about 50° C., more preferably about 20° C. to abut 30° C., and most preferably about 25° C.

After completion of the reaction for obtaining the reaction mixture comprising the protected cefdinir, the reaction mixture may be extracted with a water immiscible organic solvent to remove impurities, such as reactants not consumed during the reaction. Suitable water immiscible organic solvents include dichloromethane, C₄ to C₈ ethers and C₄ to C₇ esters or ketones. Extraction may be carried out by creating a biphasic mixture and physically stirring the two phases to facilitate moving of the impurities into the organic phase.

After the biphasic mixture is obtained, the biphasic mixture is separated and the pH of the aqueous phase is adjusted to a basic pH by addition of a base. Preferably, the pH is adjusted to about 7 to about 9, more preferably, to about 8 to about 8.5. Preferably, the buffer is NH₄Cl.

Preferably, prior to the recovering of the Cs salt, the aqueous layer containing a base in the presence of a buffer and a source of potassium or cesium to obtain cefdinir potassium salt or cesium salt is cooled and/or seeded. Preferably, the cooling is to a temperature of about 5° C. to about 15° C. The precipitate of cefdinir potassium salt or cesium salt may be recovered by conventional techniques. Preferably, the recovering is by filtration. Preferably, the precipitate is a crystalline cesium/potassium salt, which is easy to handle and can be used with ease in a manufacturing process on a commercial scale (i.e. a batch of 0.5 Kg or more) to prepare cefdinir. Optionally, the crystalline potassium salt is potassium salt form K. Cefdinir Form K is characterized by an x-ray diffraction pattern having peaks at 8.2°, 11.1°, 22.4°, 23.7°, 24.2°, and 26.3° 2-theta ±0.2° theta. Form K may be further characterized by an x-ray diffraction pattern having peaks at 13.5°, 14.5°, 15.4°, 16.1°, 18.2°, 19.5°, 20.8°, 26.7°, and 27.3° 2-theta ±0.2° theta.

The cesium or potassium salt is then converted to cefdinir by use of an acid. A solution of the cesium or potassium salt may be prepared in water or a mixture of water and water miscible organic solvent. Preferably, the solution of the cesium salt is prepared in water. Impurities from the solution may be removed by use of active carbon, a chelating agent and a filter. Preferably, the acid is added to obtain a pH of about 1 to about 4. Preferably, the acid is selected from the group consisting of: hydrochloric acid and sulfuric acid. Preferably, when the salt is cesium salt, the acid is sulfuric acid. The temperature of the solution may also be decreased or the solution seeded to further induce crystallization. A suitable temperature is about 5° C. to about 15° C.

Preferably, the obtained cefdinir is crystalline. Optionally, the obtained cefdinir is crystalline cefdinir Form A or crystalline cefdinir Form B.

The cefdinir obtained preferably has a purity of at least about 90 to about 100% as area percentage HPLC, preferably at least about 95%, more preferably at least about 97%, even more preferably at least about 99%, and most preferably at least about 99.5%.

While the present invention is described with respect to particular examples and preferred embodiments, it is understood that the present invention is not limited to these examples and embodiments. The present invention, as claimed, therefore includes variations from the particular examples and preferred embodiments described herein, as will be apparent to one of skill in the art.

### EXAMPLES

### Example 1

### Preparation of Crystalline Cefdinir Via the Potassium Salt

7-Amino-3-vinyl-3-cephem-4-carboxylic acid (“7-AVNA,” 100 g, 0.4419 mol) was added to tetrahydrofuran (1000 mL) followed by O-acetyl thioester (i.e. the compound of Formula I wherein Z is acetyl) (180 g, 0.4793 mol) and water (500 mL) with stirring. The reaction mass was cooled to 15° C. to 20° C. To this reaction mixture, triethylamine (62 mL) was added slowly at a pH of about 8.0-8.2. Stirring was continued and the progress of the reaction was monitored by qualitative HPLC until 7-AVNA was less than 1%. At this stage methylene dichloride (1000 mL) was added and the reaction mixture was stirred for 15 min at 20° C. to 25° C. Water (1000 ml) was added to the reaction mass and stirred for 15 min at 20° C. to 25° C. The aqueous layer as separated and extracted with methylene chloride (500 mL). Thereafter, ammonium chloride (66 g) was added to the aqueous part in one lot at 20° C. to 25° C. and the pH was maintained between 8.0 to 8.2 by addition of 20% w/v aqueous potassium carbonate solution. The progress of the reaction was monitored by qualitative HPLC until O-acetyl cefdinir was less than 0.5% by area. After completion of hydrolysis reaction, crystalline cefdinir potassium salts precipitated. (Seeding may be necessary to precipitate cefdinir potassium salt). The mixture was stirred for one hour and thereafter, cooled to 5° C. to 10° C. and maintained at the temperature for one hour. The precipitate was collected by filtration and the crystals were washed with a solution of 1:1 acetone:water. The product was dried under atmospheric pressure until the moisture content was about 14.7% w/w. Cefdinir potassium salt Form K (135.2 g) was obtained in 99.0% purity (by HPLC).

The cefdinir potassium (15 g) was dissolved in water (450 ml) at 25° C. to 30° C. The solution was treated with active carbon (1.5 g) and EDTA (0.15 g), and the mixture was stirred for 15-30 minutes. The solution was filtered through celite and the pH was adjusted to 1.8 to 2.4. A precipitate formed, was collected, and identified as crystalline cefdinir Form A (yield 11.3 g, HPLC 99.5%).

### Example 2

### Preparation of Crystalline Cefdinir Via the Potassium Salt

7-Amino-3-vinyl-3-cephem-4-carboxylic acid (“7-AVNA,” 100 g, 0.4419 mol) was added to tetrahydrofuran (1000 mL) followed by O-acetyl thioester (180 g, 0.4793 mol) and water (500 mL) with stirring. The reaction mass was cooled to 15° C. to 20° C. To this reaction mixture, triethylamine (62 mL) was added slowly at a pH of about 8.0-8.2. Stirring was continued and the progress of the reaction was monitored by qualitative HPLC until 7-AVNA was less than 1%. At this stage methylene dichloride (1000 mL) was added and the reaction mixture was stirred for 15 min at 20° C. to 25° C. Water (1000 ml) was added to the reaction mass and stirred for 15 min at 20° C. to 25° C. The aqueous layer as separated and extracted with methylene chloride (500 mL). Thereafter, ammonium chloride (66 g) was added to the aqueous part in one lot at 20° C. to 25° C. and the pH was maintained between 8.0 to 8.2 by addition of 20% w/v aqueous potassium carbonate solution. The progress of reaction was monitored by qualitative HPLC until O-acetyl cefdinir was less than 0.5% by area. After completion of the hydrolysis reaction, crystalline cefdinir potassium salts precipitated. (Seeding may be necessary to precipitate cefdinir potassium salt). The mixture was stirred for one hour and thereafter, cooled to 5° C. to 10° C. and maintained at the temperature for one hour. The precipitate was collected by filtration and the crystals were washed with a solution of 1:1 acetone:water. The product was dried under atmospheric pressure until the moisture content was about 14.7% w/w. Cefdinir potassium salt Form K (135.2 g) was obtained in 99.0% purity (by HPLC).

The cefdinir potassium (15 g) was dissolved in water (450 mL) at 25° C. to 30° C. The solution was treated with active carbon (1.5 g) and EDTA (0.15 g) and the mixture was stirred for 15-30 minutes. The solution was filtered through celite and the pH was adjusted to 1.8 to 2.4 at 8° C. to 12° C. The solution was stirred and a precipitate was collected and identified as crystalline cefdinir Form-B (yield 11.3 g, HPLC 99.5%).

### Example 3

### Preparation of Crystalline Cefdinir Via the Cesium Salt

100 g of 7-amino-3-vinyl-3-cephem-4-carboxylic acid (7-AVNA, 0.4419 mol) was added to 1000 ml of tetrahydrofuran followed by 180 g of O-acetyl thioester (0.4793 mol) and 500 ml of water with stirring. The reaction mass was cooled to 15-20° C. To this reaction mixture, 62 ml of triethylamine was added slowly at a pH of about 8.0-8.2. Stirring was continued and progress of the reaction was monitored by qualitative HPLC until 7-AVNA was less than 1%. At this stage 1000 ml of methylene dichloride was added and stirred for further 15 min at 20-25° C. 250 ml of water was added and the reaction mass was stirred for 15 min at 20-25° C. The layers were separated the aqueous layer was further extracted by 500 ml methylene chloride. Thereafter, 66 g of ammonium chloride was added to the aqueous part in one lot at 20-25° C. and the pH of the hydrolysis mass was maintained at 7.8 to 8.2 by addition of 40% w/v aqueous cesium carbonate solution. The progress of reaction was monitored by qualitative HPLC until O-acetyl cefdinir was less than 0.5% after completion of hydrolysis reaction mass the precipitation of crystalline cefdinir. Cesium salts are observed (if precipitation was not observed after clear solution of reaction mass, the mass was seeded with Cefdinir Cesium salt. At that point, the reaction mixture was further stirred for another hour, and then cooled to 5° C. to 10° C. for another hour). The slurry was filtered and the product was washed with acetone. The product was dried under atmospheric pressure until moisture content of 7.9% w/w. 146 g of product was obtained with 99.0% purity (by HPLC).

The cefdinir cesium salt (100 g) was dissolved in water (2500 ml) at 25 to 30° C. Active carbon (10 g) and EDTA (1.0 g) were added to the resulting solution and mixture was stirred for 15-30 minutes at 25 to 30° C. The product was filtered through celite and the pH of the clear solution was adjusted to 2.2 to 2.5 at 25-30° C. by addition of 10% hydrochloric acid and stirred at that temperature to obtain crystalline cefdinir form-A (yield 74 g, HPLC 99.8%).

### Example 4

### Preparation of Crystalline Cefdinir Via the Cesium Salt

100 g of 7-amino-3-vinyl-3-cephem-4-carboxylic acid (7-AVNA, 0.4419 mol) was added to 1000 ml of tetrahydrofuran followed by 180 g of O-acetyl thioester (0.4793 mol,) and 500 ml of water with stirring. The reaction mass was cooled to 15-20° C. To this reaction mixture, 62 ml of triethylamine was added slowly at a pH if about 8.0-8.2. Stirring was continued and the progress of the reaction was monitored by qualitative HPLC until 7-AVNA was less than 1%. At this stage 1000 ml of methylene dichloride was added and stirred for another 15 min at 20-25° C. 250 ml of water was added and the reaction mass was stirred for 15 min at 20-25° C. The layers were separated and the aqueous layer was further extracted by 500 ml methylene chloride. Thereafter, 66 g of ammonium chloride was added to the aqueous part in one lot at 20-25° C. and the pH of the hydrolysis mass was maintained at 7.8 to 8.2 by addition of 40% w/v aqueous cesium carbonate solution. The progress of the reaction was monitored by qualitative HPLC until O-acetyl cefdinir was less than 0.5 after completion of hydrolysis reaction mass the precipitation of crystalline cefdinir Cesium salts is observed. (if precipitation was not observed after clear solution of reaction mass, the mass was seeded with cefdinir cesium salt. At that point, the reaction mixture was further stirred for another one hour, and then cooled to 5° C. to 10 ° C. for another hour). The temperature is maintained for another hour. The slurry was filtered and the product was washed with acetone. The product was dried under atmospheric pressure until moisture content of 7.9% w/w. 146 g of product was obtained with 99.0% purity (by HPLC).

The cefdinir cesium salt (100 g) was dissolved in water (2500 ml) at 25 to 30° C. Active carbon (10 g) and EDTA (1.0 g) were added to the resulting solution and the mixture was stirred for 15-30 minutes at 25 to 30° C. It was filtered through celite and the pH of the clear solution was adjusted to 2.2 to 2.5 at 8 to 12° C. by addition of 10% hydrochloric acid and stirred at that temperature to obtain crystalline cefdinir Form-B (yield 74 g, HPLC 99.5%).

### Example 5

### Preparation of Crystalline Cefdinir Via the Cesium Salt

10 grams of cefdinir cesium salt was dissolved in 300 ml water at 20° C.-30° C. 1.0 grams of Charcoal and 0.1 grams of EDTA were added into the solution, and the solution was stirred for 30 minutes. After that, the reaction mixture was filtered to remove the carbon, by high flow bed washed hi flow bed with water. Thereafter, the clear filtrate was taken to another flask, cooled to 10° C. to 15° C. 5% of aqueous HCl were added to the solution to get pH of 0.5 to 0.6. Solid cefdinir precipitated out from the solution. The solution was further stirred at that point for one hour to obtain slurry. Thereafter, the slurry was filtered to obtain a wet cake. The wet cake was suspended in 200 ml of water at 30° C.-35° C., stirred for one hour and filtered. Then, the cake was washed with water, till absence of chloride in mother liquor. The wet cake was unloaded and dried under reduced pressure at 40° C. until obtaining constant weight. Yield: 4.8 grams, Purity 99.6%, water content 7.2%.

### Example 6

### Preparation of Crystalline Cefdinir Via the Cesium Salt

10 grams of cefdinir cesium salt was dissolved in 300 ml water at 20° C.-30° C. Charcoal 1.0 grams and 0.1 grams EDTA were added to the solution, and the solution was stirred for 30 minutes. The carbon was filtered on high flow bed washed hi flow bed with water. Thereafter, the clear filtrate was taken to another flask, cooled to 10° C. to 15° C. 5% aqueous HCl were added to the solution to get pH of 0.5 to 0.6. Solid cefdinir precipitated out from the solution. After that, the pH was adjusted to 2.2 to 2.5 by adding aqueous ammonia and was stirred at that point for one hour, to obtain a slurry. The slurry was filtered to obtain a wet cake, the wet cake was suspended in 200 ml of water at 30° C.-35° C., stirred for one hour and filtered. The cake was washed with water, till absence of chloride in mother liquor. The wet cake was unloaded and dried under reduced pressure at 40° C. until obtaining constant weight. Yield: 6.1 grams, Purity 99.3%, water content 6.9%.

We claim:

1. A process for preparing cefdinir comprising:
(a) reacting a protected thioester of Formula I: 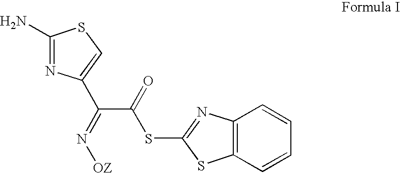
, 7-amino-3-vinyl-3-cephem-4-carboxylic acid and at least one organic base in the presence of water and a water-miscible organic solvent to form protected cefdinir;
(b) converting the protected cefdinir to a cefdinir salt selected from the group consisting of cefdinir potassium and cefdinir cesium; and
(c) converting the cefdinir salt to cefdinir;
wherein Z represents an oxime protecting group.

2. The process of claim 1, wherein the cefdinir salt obtained in step b) is recovered prior to step c).

3. A process for preparing cefdinir comprising:
(a) reacting a thioester of Formula I 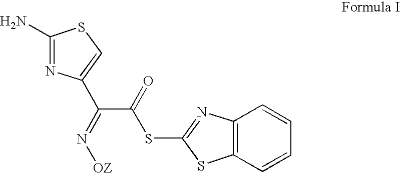
with 7-amino-3-vinyl-3-cephem-4-carboxylic acid and at least one organic base in the presence of water and a water-miscible organic solvent to obtain a reaction mixture comprising protected cefdinir;
(b) treating the reaction mixture with a base in the presence of a buffer and a source of potassium or cesium ions to obtain cefdinir potassium salt or cesium salt to form a mixture;
(c) recovering the salt from the mixture;
(d) dissolving the salt in water or a mixture of water and a water miscible organic solvent to form a solution; and
(e) adding an acid to the solution to obtain cefdinir, wherein Z represents an oxime protecting group.

4. The process of claim 3, wherein the oxime protecting group is selected from the group consisting of: acetyl, 2-amino thiazole and tetrahydropyranyl.

5. The process of claim 3, wherein the water-miscible organic solvent is selected from the group consisting of tetrahydrofuran, ethanol, methanol, propanol, isopropanol, N, N dimethyl formamide, dimethyl acetamide, acetonitrile and mixtures thereof.

6. The process of claim 3, wherein the water-miscible organic solvent is tetrahydrofuran.

7. The process of claim 3, wherein the ratio of the water-miscible solvent to water is about 1:1 to about 10:1 (v:v).

8. The process of claim 3, wherein the ratio of the water-miscible solvent to water is about 2.5:1 (v:v).

9. The process of claim 3, wherein the organic base is an amine base.

10. The process of claim 9, wherein the amine base is a C₃ to C₁₂ amine.

11. The process of claim 10, wherein the C₃-C₁₂ amine is selected from the group consisting of diethylamine, triethylamine, diisopropylamine, tri-n-butylamine, triethylenediamine, and pyridine.

12. The process of claim 10, wherein the amine base is a C₃-C₉ amine.

13. The process of claim 12, wherein the C₃-C₉ amine is triethylamine.

14. The process of claim 3, wherein the reaction mixture comprising the protected cefdinir is stirred.

15. The process of claim 14, wherein the reaction mixture is stirred for about 2 to about 8 hours.

16. The process of claim 15, wherein the reaction mixture is stirred for about 4 to about 6 hours.

17. The process of claim 3, wherein the reaction of step (a) is carried out at a temperature of about 0° C. to about 50° C.

18. The process of claim 17, wherein the temperature is about 20° C. to abut 30° C.

19. The process of claim 18, wherein the temperature is about 25° C.

20. The process of claim 3, wherein the reaction mixture comprising the protected cefdinir is extracted with a water immiscible organic solvent.

21. The process of claim 20, wherein the water immiscible organic solvent is dichloromethane, a C₄ to C₈ ether, or a C₄ to C₇ ester or ketone.

22. The process of claim 20, wherein the extraction is carried out by creating a biphasic mixture having an aqueous and an organic phase and physically stirring the two phases to facilitate moving of the impurities into the organic phase.

23. The process of claim 22, wherein the aqueous phase is separated from the biphasic mixture and the pH of the aqueous phase is adjusted by addition of a base.

24. The process of claim 23, wherein the pH is adjusted to about 7 to about 9.

25. The process of claim 24, wherein the pH is adjusted to about 8 to about 8.5.

26. The process of claim 3, wherein the buffer is ammonium chloride.

27. The process of claim 3, wherein the mixture of step (b) is cooled to promote crystallization of the cefdinir potassium or cesium salt.

28. The process of claim 27, wherein the mixture of step (b) is cooled to a temperature of about 5° C. to about 15° C.

29. The process of claim 3, wherein the mixture of step (b) is seeded to promote crystallization of the cefdinir potassium or cesium salt.

30. The process of claim 3, wherein the recovered salt is cefdinir potassium salt form K characterized by an X-ray diffraction pattern having peaks at 8.2°, 11.1°, 22.4°, 23.7°, 24.2°, and 26.3°, 2-theta ±0.2° theta.

31. The process of claim 30, wherein the salt is further characterized by an X-ray diffraction pattern having peaks at 13.5°, 14.5°, 15.4°, 16.1°, 18.2°, 19.5°, 20.8°, 26.7°, and 27.3° 2-theta ±0.2° theta.

32. The process of claim 3, wherein the salt in step (d) is dissolved in water.

33. The process of claim 3, wherein the solution of step (d) is treated with active carbon or a chelating agent.

34. The process of claim 3, wherein the acid is added to obtain a pH of about 1 to about 4.

35. The process of claim 3, wherein the acid is selected from the group consisting of hydrochloric acid and sulfuric acid.

36. The process of claim 3, wherein the salt is the cesium salt.

37. The process of claim 36, wherein the acid is sulfuric acid.

38. The process of claim 3, wherein the solution of step (e) is cooled to promote crystallization of cefdinir.

39. The process of claim 38, wherein the solution of step (e) is cooled to a temperature of about 5° C. to about 15° C.

40. The process of claim 3, wherein the solution of step (e) is seeded to promote crystallization of cefdinir.

41. The process of claim 3, wherein the obtained cefdinir is crystalline.

42. The process of claim 41, wherein the obtained cefdinir is crystalline cefdinir Form A or crystalline cefdinir Form B.

43. The process of claim 3, wherein the obtained cefdinir has a purity of at least about 90 to about 100% as area percentage HPLC.

44. The process of claim 43, wherein the obtained cefdinir has a purity of at least about 95% as area percentage HPLC.

45. The process of claim 44, wherein the obtained cefdinir has a purity of at least about 97% as area percentage HPLC.

46. The process of claim 45, wherein the obtained cefdinir has a purity of at least about 99% as area percentage HPLC.

47. The process of claim 46, wherein the obtained cefdinir has a purity of at least about 99% as area percentage HPLC.

48. The process of claim 3, wherein the salt is cefdinir potassium salt.

49. The process of claim 3, wherein the salt is cefdinir cesium salt.
